(19) United States
(12) Patent Application Publication
(10) Pub. No.: US 2026/0097190 A1
(43) Pub. Date: Apr. 9, 2026

(54) DRUG DELIVERY DEVICE

(71) Applicant: LIBERUM HEALTH LTD, London (GB)

(72) Inventors: Alexander SEIFALIAN, London (GB); Amelia SEIFALIAN, London (GB); Chayma NOUAJAA, London (GB); Alena VDOVCHENKO, London (GB); Karolina AFORS, London (GB); Hana SALUSSOLIA, London (GB)

(73) Assignee: LIBERUM HEALTH LTD, London (GB)

(21) Appl. No.: 19/120,894

(22) PCT Filed: Oct. 16, 2023
(86) PCT No.: PCT/GB2023/052681

§ 371 Date: Apr. 14, 2025

(30) Foreign Application Priority Data
Oct. 14, 2022 (GB) 2215216.9

(51) Int. Cl. A61M 31/00

(52) U.S. Cl. CPC A61M 31/002; A61M 2205/0244; A61M 2207/00; A61M 2210/1475

(57) ABSTRACT

A drug delivery device including a porous polymeric material for modulating the delivery of the drug, and a method of manufacture thereof includes: a charged drug delivery device, and a method of manufacture thereof, wherein the charged drug delivery device includes the drug delivery device and a drug, wherein the drug is encapsulated by the drug delivery device, the charged drug delivery device for use in administering a drug, and a kit-of-parts comprising the drug delivery device and a drug for encapsulation in the drug delivery device.

### FIELD

The present invention relates to the field of medical devices, more particularly, to the field of drug delivery devices and methods for their production.

### BACKGROUND

The delivery of drugs by non-oral route avoids gastro-intestinal drug absorption and hepatic first pass metabolism. Body cavities, such as the vagina, may be used to deliver drugs via direct absorption into the blood stream. The vagina is a viscoelastic muscular tube with epithelium cells lining with a surface area of about 85 cm² and highly vascularised, meaning that the anatomy and physiology of the vagina allows smaller doses of drug to be administered as a result of the effective and direct delivery of drugs by this means.

To take an example, dysmenorrhoea, a specialist term used to describe menstrual pain, is estimated to affect up to 90% of women of reproductive age, often without an identifiable cause. There are some known causes of pelvic pain, including endometriosis, which can be disabling, resulting in time off work and great impact on mental and social wellbeing. The medical management options for dysmenorrhoea come with a host of systemic side-effects. For example, anti-inflammatory based analgesia greatly increases risks of stomach ulcers and further gastro-intestinal upset. Paracetamol is also an option; however it is often not found to be effective for long durations and it presents high risk of overdose. Cannabis-based products for medicinal use (CBPM) have recently been exploited and investigated for the application as an alternative treatment strategy to manage dysmenorrhoea. CBPMs provide a more natural and less harmful treatment option than the aforementioned forms of analgesia. There is no documented amount at which CBPM results in drug overdose, it is understood to be extremely safe, even at higher doses. If prescribed and delivered in a safe and controlled manner, CBPM can manage dysmenorrhoea.

Thus, there is need for a safe and effective non-oral drug delivery device which allows smaller doses of drug to be administered, allows drugs to be delivered over a longer period of time and which reduces systemic side-effects that each drug may cause. Thus, it is a problem of the present invention to provide a drug delivery device for improved delivery of a drug, particularly improved localised drug delivery.

It is further a problem of the present invention to provide a drug delivery device, preferably a drug delivery device for insertion into a body cavity, which is non-toxic (preferably, neither locally acute non-toxic nor systemically acute non-toxic), and is adaptable/conformable to the shape of the body cavity in which it is inserted.

Moreover, many drug delivery devices tend to be used only once before being discarded, whereby they end up in landfill or in aqueous environments such as the sea. Therefore, it is also a problem of the present invention to provide a drug delivery device which is biodegradeable in landfill or aqueous environments such as freshwater, brackish water or seawater, yet is also recyclable. Furthermore, it is a problem of the present invention to provide a drug delivery device which, after degradation in landfill or aqueous environments leaves no toxic substances behind,

### BRIEF DESCRIPTION

The present invention relates to a drug delivery device (i.e. a device suitable for delivery of a drug) comprising a porous polymeric material for modulating the delivery of said drug, said porous polymeric material comprising at least one polymer comprising a carbon nanoparticle covalently bonded to a polyurethane moiety, preferably wherein said polymer comprises a carbon nanoparticle covalently bonded to a polyurethane moiety which comprises at least one polyester moiety which has the same structure as the polyester moiety comprised in polycaprolactone diol, more preferably wherein said polymeric material is BioHastalex®.

The present invention also relates to a method of manufacture of a drug delivery device (i.e. a device suitable for delivery of a drug) comprising a porous polymeric material for modulating the delivery of said drug, said porous polymeric material comprising at least one polymer comprising a carbon nanoparticle covalently bonded to a polyurethane moiety, wherein said method comprises the following steps:
(i) mixing said porous polymeric material resin precursor with a porogen to form a suspension;
(ii) forming a layer of said suspension; and
(iii) coagulating and curing the layer formed in step (ii), preferably wherein said polymer comprises a carbon nanoparticle covalently bonded to a polyurethane moiety which comprises at least one polyester moiety which has the same structure as the polyester moiety comprised in polycaprolactone diol, more preferably wherein said polymeric material is BioHastalex®.

In addition, the present invention relates to a charged drug delivery device (i.e. a drug delivery device, as defined herein, which is charged or loaded with said drug) comprising:
(a) a drug delivery device as defined herein; and
(b) a drug, wherein said drug is encapsulated by the porous polymeric material comprised in said drug delivery device.

Moreover, the present invention relates to a method of manufacture of a charged drug delivery device, wherein said method comprises encapsulating a drug inside the porous polymeric material comprised in the drug delivery device as defined herein.

Furthermore, the present invention relates to a kit-of-parts comprising:
(a) a drug delivery device as defined herein or manufactured according to the method as defined herein; and
(b) a drug for encapsulation in the porous polymeric material comprised in said drug delivery device.

The present invention additionally relates to a charged drug delivery device for use in administering a drug, wherein said charged drug delivery device is the charged drug delivery device as defined herein.

### FIGURES

FIG. 1 shows an illustration of a typical instrument used in the manufacture of a drug delivery device according to an embodiment of the present invention.

FIG. 2 shows an illustration of a drug delivery device according to an embodiment of the present invention at varying stages of fabrication A) the empty drug delivery device pre-loading; B) the drug delivery device once charged with drug; C) close-up view of the reservoir inserted into the drug delivery device; D) an embodiment of the finished drug delivery device in a pessary or tampon-like device (TLD) form; E) an embodiment of the finished drug delivery device in a toroidal (ring) pessary form.

FIG. 3 shows an illustration of the drug delivery device according to certain embodiments of the invention: A) exploded view of the final drug delivery device in pessary or TLD form; B) exploded view of the final drug delivery device incorporated into the tip of a tampon.

FIG. 4 shows an illustration of the drug delivery device according to certain embodiments of the invention: A) a cut-through view of the final drug delivery device in a toroidal (ring) pessary form; B) close-up view of the poloidal cross-section of FIG. 4A.

FIG. 5 shows a scanning electron microscopy (SEM) image of porous polymeric material scaffold having different porosities according to the present invention: A) pore size 60 um; B) pore size 100 um

FIG. 6 shows a series of illustrations of the drug delivery device according to certain embodiments of the present invention in use: A) illustrates the drug delivery device in a pessary form when installed; B) illustrates the drug delivery action of the drug delivery device in a pessary form.

FIG. 7 shows illustrates the shape-memory capability of an embodiment of the drug delivery device according to the present invention.

FIG. 8 shows a graph demonstrating the drug-release profile of a drug delivery device according to an embodiment of the present invention.

FIG. 9 shows a graph demonstrating the in vivo diclofenac release profile in the plasma of nulliparous and nonpregnant adult female rats intravaginally inserted with a drug delivery device according to an embodiment of the present invention charged with 70 mg diclofenac.

FIG. 10 shows a graph demonstrating the in vivo cannabidiol (CBD) release profile in the plasma of nulliparous and nonpregnant adult female rats intravaginally inserted with a drug delivery device according to an embodiment of the present invention charged with 70 mg CBD.

### DETAILED DESCRIPTION

The present invention relates to a drug delivery device (i.e. a device suitable for delivery of a drug), as well as to a method for its manufacture.

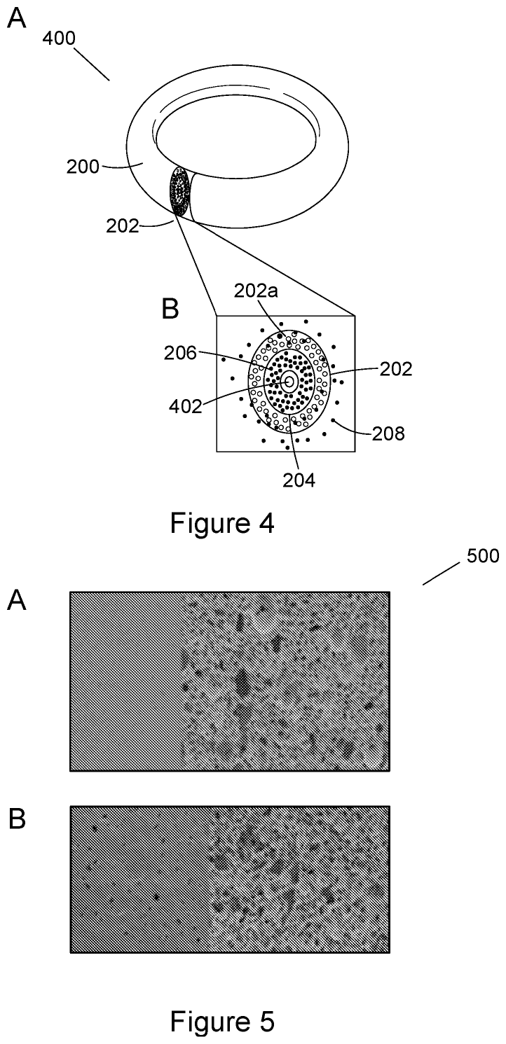

Said device is suitable for delivery of a drug to a tissue of the human or animal body. Preferably, said device comprises a wall separating an internal space of said drug delivery device from the outside of said drug delivery device, wherein said internal space is suitable for containing a drug therein and releasing said drug therefrom. In a preferred embodiment of the present invention, said device is a suppository (i.e. configured for insertion inside the human or animal body and delivery of a drug thereto, preferably for insertion inside a vaginal, anal, urethral, buccal or nasal cavity). In another preferred embodiment of the present invention, said device is selected from the group consisting of a pessary (FIGS. 3A and 4), tampon (FIG. 3B), menstrual cup and any other gynaecological device. More preferably, said device is selected from a pessary or a tampon.

Said device comprises a porous polymeric material for modulating the delivery of said drug. Said polymeric material comprises at least one polymer comprising a carbon nanoparticle covalently bonded to a polyurethane moiety. Preferably, said polymer comprises a carbon nanoparticle covalently bonded to a polyurethane moiety via a urea moiety.

The carbon nanoparticle is selected from the group consisting of graphene oxide, reduced graphene oxide, a carbon nanotube, fullerene, graphite, graphene, diamond, a carbon dot, functionalised graphene oxide, a functionalised carbon nanotube, functionalised fullerene, functionalised graphite, functionalised graphene, functionalised diamond, a functionalised carbon dot and a functionalised graphene oxide to which silica nanoparticles are attached. Preferably the carbon nanoparticle is selected from the group consisting of graphene, functionalised graphene, or functionalised graphene oxide.

The term “functionalised” means functionalised with at least one moiety independently selected from the group consisting of a carboxylic acid, tertiary alcoholic, epoxide, primary alcoholic or primary amino moiety (ignoring the functionalisation provided by the carbon nanoparticle being covalently bonded to said polyurethane moiety). Preferably “functionalised” means functionalised with at least one moiety independently selected from the group consisting of a primary alcoholic or primary amino moiety. Graphene oxide comprises carboxylic acid, tertiary alcoholic, epoxide and/or primary alcoholic groups, wherein the carboxylic acid groups can be found on the edge of the graphene sheets in the plane of the sheets, such that graphene oxide is a functionalised graphene in the sense described herein. Thus, when the carbon nanoparticle is functionalised graphene oxide, it may be functionalised with at least one moiety, each moiety being independently selected from the group consisting of a carboxylic acid, tertiary alcoholic, epoxide, primary alcoholic or primary amino group, preferably a primary amino group, more preferably wherein the primary amino groups are in the plane and on the edge of the graphene sheets. Similarly, when the carbon nanoparticle is functionalised graphene, it may be functionalised with at least one moiety, each moiety being independently selected from the group consisting of a carboxylic acid, tertiary alcoholic, epoxide, primary alcoholic or primary amino group, preferably a primary amino group, more preferably wherein the primary amino groups are in the plane and on the edge of the graphene sheets.

The polyurethane moiety preferably comprises at least one polycarbonate moiety, at least one polyester moiety, at least one polyether moiety or at least one polyalkadiene moiety. More preferably the polyurethane moiety comprises at least one polycarbonate moiety which has the same structure as the polycarbonate moiety comprised in Eternacoll® PH-200 (UBE) or Perstorp Oxymer™ M-112 (Perstorp catalogue HS number: 390740), at least one polyester moiety which has the same structure as the polyester moiety comprised in polycaprolactone diol (polycaprolactone oxydiethylene ester, average Mn˜2,000; Sigma-Aldrich catalogue number 189421), at least one polyether moiety which has the same structure as the polyether moiety comprised in Invista Terethane® PTMEG 2000 (polytetramethylene ether glycol, average Mn˜2,000), or at least one polyalkadiene moiety which has the same structure as the polyalkadiene moiety comprised in Cray Valley Krasol® LBH 2000 (hydroxyl-terminated polybutadiene). Even more preferably, said polyurethane moiety comprises a poly(hexamethylene carbonate) or a poly(caprolactone) moiety, the latter of which is readily biodegradable.

In one embodiment, said polymer is made by a method comprising combining or mixing a functionalised carbon nanoparticle with a polyol and adding a diisocyanate thereto to form a pre-polymer solution. The pre-polymer solution is subjected to chain extension in the presence of a diamine to yield a polymer solution (resin precursor). The polymer solution (resin precursor) is subsequently cured to form a polymeric material comprising said polymer. The polymeric material may have surface hydrophobicity or hydrophilicity.

In a particularly preferred embodiment, the polymer solution (resin precursor) is made according to any of the methods disclosed in WO2019/008381 A1, in particular any one of Examples 2, 16 and 17 of WO2019/008381 A1. Said methods may be adapted to use different starting materials, including, for example, replacing the polycarbonate polyol referred to therein with a polycaprolactone diol (e.g. polycaprolactone oxydiethylene ester, average Mn˜2,000; Sigma-Aldrich catalogue number 189421). Likewise, in a particularly preferred embodiment, the porous polymeric material is made from said polymer solution according to the methods disclosed in Example 18 of WO2019/008381 A1 used for formation of a porous scaffold. Said methods may be adapted to use different starting materials, including, for example, replacing the porogen referred to therein with an alternative porogen such as sodium chloride.

In one embodiment, said polymeric material is the polymeric material known as Hastalex®. The Hastalex® polymeric material is made by curing Hastalex® polymer solution (resin precursor), as described herein. Briefly, Hastalex® polymer solution is made by mixing functionalised graphene oxide nanoparticles, namely a graphene oxide functionalised with at least one primary amine group, with a poly(hexamethylene carbonate) diol [e.g. Eternacoll® PH-200 (UBE catalogue number) or Perstorp 30 Oxymer™ M-112 (Perstorp catalogue HS number: 390740)], and adding 4,4′-methylenebis(phenyl isocyanate) thereto to form a pre-polymer solution which is subsequently subjected to chain extension in the presence of ethylenediamine and diethylamine or dibutylamine in dimethylacetamide. Preferably, said Hastalex® polymer solution may be obtained according to Example 17 of WO2019/008381 A1, while the corresponding polymeric material may be obtained according to Example 18 of WO2019/008381 A1.

In another embodiment, said polymeric material is the polymeric material known as BioHastalex®. The BioHastalex® polymeric material is made by curing BioHastalex® polymer solution (resin precursor), as described herein. Briefly, BioHastalex® polymer solution is made by mixing functionalised graphene oxide nanoparticles, namely a graphene oxide functionalised with at least one primary amine group, with a polycaprolactone diol (e.g. polycaprolactone oxydiethylene ester, average Mn˜2,000; Sigma-Aldrich catalogue number 189421), and adding 4,4′-methylenebis(phenyl isocyanate) thereto to form a pre-polymer solution which was subsequently subjected to chain extension in the presence of ethylenediamine and diethylamine or dibutylamine in dimethylacetamide. BioHastalex® is biodegradable, recyclable and non-toxic (i.e. biocompatible). Preferably, said BioHastalex® polymer solution may be obtained according to Example 17 of WO2019/008381 A1 by replacing the polycarbonate polyol referred to therein with a polycaprolactone diol (e.g. polycaprolactone oxydiethylene ester, average Mn˜2,000; Sigma-Aldrich catalogue number 189421), while the corresponding polymeric material may be obtained according to Example 18 of WO2019/008381 A1 using said BioHastalex® polymer solution as the composite material of Example 17 of WO2019/008381 A1. The BioHastalex® polymer solution and the corresponding polymeric material produced therefrom are biodegraded when in landfilled or seawater over two years. When used as the polymeric material in the drug delivery device according to the present invention, this reduces significantly microplastic pollution in comparison to currently used pessary devices which are made of silicone or vinyl, both materials being non-biodegradable and not environmentally friendly. Conventional pessary rings are changed every 3 to 6 months and are thrown away after use.

In forming Hastalex® and BioHastalex®, said graphene oxide is functionalised by converting at least one carboxylic acid moiety present thereon into the corresponding primary amine or methyleneamine moieties. Therefore, when said polymeric material is the polymeric material known as Hastalex®, said polymeric material comprises at least one polymer comprising a carbon nanoparticle covalently bonded to a polyurethane moiety which comprises at least one polycarbonate moiety which has the same structure as the polycarbonate moiety comprised in Eternacoll® PH-200 (UBE) or Perstorp Oxymer™ M-112 (Perstorp catalogue HS number: 390740). Similarly, when said polymeric material is the polymeric material known as BioHastalex®, said polymeric material comprises at least one polymer comprising a carbon nanoparticle covalently bonded to a polyurethane moiety which comprises at least one polyester moiety which has the same structure as the polyester moiety comprised in polycaprolactone diol (e.g. polycaprolactone oxydiethylene ester, average Mn˜2,000; Sigma-Aldrich catalogue number 189421).

Said polymeric material may also comprise polymers other than those disclosed herein, including polymers which do not comprise a carbon nanoparticle. Thus, in one embodiment, said polymeric material comprises at least one polymer, as described herein, and at least a second polymer comprising a polyurethane moiety, as described herein.

Said polymeric material is porous and therefore comprises pores (FIGS. 5A and 5B). Said pores are preferably filled with air and/or water and/or a drug or a drug dispersed in a hydrogel matrix. Said pores preferably have a size (equivalent spherical diameter) falling between 50 nm and 1000 μm, more preferably between 65 nm and 1000 μm, even more preferably between 1 and 500 μm. The pore sizes correspond linearly with the size of the porogen particles used to make said pores and were determined according to ISO 15901-2:2022.

Thus, the porogen used for making the pores of said porous polymeric material preferably comprises a material which is solid at between 2 and 90° C. Said solid has a particle size (equivalent spherical diameter) falling in the range of between 50 nm and 1000 μm. In a preferred embodiment of the present invention, the porogen used for making the pores of said porous polymeric material comprises a solid which has a particle size of between 65 nm and 1000 μm, even more preferably between 1 and 500 μm. Said solid porogen is made by grinding the solid material used for making the porogen using a mill, after which at least two sieves were used to separate the ground solid material into solid material falling in size between the above ranges by agitation according to ISO 2591-1:1988, e.g. separation of porogen into a size falling between 50 nm and 1000 μm was achieved using a sieve of 50 nm and a sieve of 1000 μm. Preferably, said sieves are stainless steel sieves, more preferably sieves comprised in an electric sieve shaker.

The solid porogen is soluble in a solvent which the polymeric material is not soluble in, preferably a water-soluble solid porogen, more preferably a water-soluble salt or sugar, even more preferably a solid porogen selected from the group consisting of sucrose, sodium chloride, sodium hydrogen carbonate, sodium carbonate, sodium sulfate, sodium phosphate, potassium chloride, potassium hydrogen carbonate, potassium carbonate, potassium sulfate, potassium phosphate, calcium chloride and magnesium chloride. Still more preferably, the porogen is a solid porogen selected from the group consisting of sucrose, sodium chloride and sodium hydrogen carbonate.

The polymer solution (resin precursor) is so-described because it comprises at least one polymer, as described herein, dissolved in it. In addition, said polymer solution may also comprise a solvating agent such as N,N-dimethylacetamide (DMAC) or dimethylsulfoxide (DMSO). Said solvating agent may be that which was employed in the formation of said polymer solution. Exposure of the polymer solution or suspension comprising said polymer solution to air at between 5° and 90° C., or to said solvent at between 2 and 90° C. may result in release of said solvating agent from said polymer solution, leaving behind nanopores (i.e. pores with internal widths of >2 nm to 100 nm) and micropores (i.e. pores with internal widths of 2 nm and less) (said pore sizes being determined as per ISO 15901-2:2022). Thus, the pores in the porous polymeric material may form from use of said porogen and/or said solvating agent.

The polymer solution may also comprise a surfactant to increase wettability of the porogen and decrease aggregation of solid porogen particles in the mixture (suspension) comprising said polymer solution and said porogen (and hence increase uniformity of pore size in the porous polymeric material). Said surfactant is preferably selected from the group consisting of: Tween-20 (Polysorbate 20), Tween-40 (Polysorbate 40), Tween-60 (Polysorbate 60), Tween-80 (Polysorbate 80), Sorbitan monolaurate, Sorbitan monostearate and Sorbitan tristearate, more preferably from Tween-20 or Sorbitan monostearate.

Removal of the porogen or liquid material during or after curing the polymer solution results in pores in the polymeric material. Preferably the porous polymeric material is polydisperse, having pores of different sizes.

The porogen used for making the pores of said porous polymeric material may be present at between 0.1 and 80 wt. % in the resin precursor (polymer solution) used for making it. In a preferred embodiment of the present invention, the porogen used for making the pores of said porous polymeric material may be present at between 1 and 40 wt. % in the resin precursor (polymer solution) used for making it. More preferably, the porogen used for making the pores of said porous polymeric material may be present at between 5 and 20 wt. % or at between 21 and 38 wt. % in the resin precursor (polymer solution) used for making it. Thus, in one extreme, the porous polymeric material is a solid with pores inside it, and in another extreme it is a sponge comprised of open cells. In practice, the porous polymeric material lies somewhere between these extremes, with a portion of the pores being open to the outside of said polymeric material and allowing drug delivery.

The porous polymeric material is made by a process comprising a step of mixing said polymeric material resin precursor (polymer solution) with a porogen to form a suspension. Said mixing may comprise any process to increase homogeneity of the mixture (suspension) comprising said polymer solution and said porogen as well as to decrease aggregation of solid porogen particles. Preferably said mixing involves agitation (e.g. stirring, swirling and/or shaking), heating and/or sonication. The resulting suspension is then coagulated and cured. Coagulation and curing is preferably achieved by exposing said suspension to either:
air at between 5° and 90° C. and optionally subsequently treated with a solvent, or
said solvent at between 2 and 90° C.,
   wherein said solvent is a solvent which the polymeric material is not soluble in, but which the porogen is soluble in. In this manner, the exposure to air at between 5° and 90° C. or to said solvent at between 2 and 90° C., coagulates and cures the polymer solution to form the polymeric material, and said solvent leaches the porogen from the suspension, creating pores. When said solvent is used for coagulation, coagulation takes place by phase inversion. Preferably said solvent is water, and said porogen is water-soluble.

Preferably the time of exposure to said solvent is a minimum of 10 seconds, more preferably between 30 seconds and 10 minutes, even more preferably between 1 and 3 minutes. Exposure to said solvent in this manner allows dissolution and leaching of porogen, and leaching of liquid material.

The porous polymeric material is preferably formed by solvent casting, wherein the suspension of said polymeric material resin precursor (polymer solution) and said porogen is coated or sprayed on a surface to form a layer thereon. Subsequent coagulation and curing, as well as leaching of the porogen from said layer, results in a layer of the porous polymeric material. In particular, the present invention relates, in one aspect, to a method of manufacture of a drug delivery device comprising a porous polymeric material for modulating the delivery of said drug, said porous polymeric material comprising at least one polymer comprising a carbon nanoparticle covalently bonded to a polyurethane moiety, wherein said method comprises the following steps:
(i) mixing said porous polymeric material resin precursor with a porogen to form a suspension;
(ii) forming a layer of said suspension; and
(iii) coagulating and curing the layer formed in step (ii), preferably by exposing said layer to air at between 5° and 90° C. or to water at between 2 and 90° C.

Further repeating this solvent casting technique n times, each time over the previously formed layer of porous polymeric material, results in a porous polymeric material having n+1 layers, wherein n is preferably a whole number selected from the group consisting of from 1 to 20. Thus, in one embodiment of the method of manufacture of the present invention said method comprises additional steps of:
(iv) forming a layer of said suspension on the porous polymeric material;
(v) coagulating and curing the layer formed in step (iv), preferably by exposing the laminate formed in step (iv) to air at between 5° and 90° C. or to water at between 2 and 90° C.; and
(vi) optionally repeating steps (vi) and (v).

More preferably, n is a whole number from 1 to 25 (i.e. where steps (i) to (iii) are carried out once and steps (iv) to (vi) are carried out from 1 to 25 times), even more preferably a whole number from 2 to 15, even more preferably a whole number from 5 to 11.

The porogen used in the method of manufacture has a particle size as defined herein. In one embodiment of the method of manufacture of the present invention, said porogen has a particle size of between 65 nm and 1000 μm.

The porogen used in the method of manufacture is present in the proportions defined herein. In one embodiment of the method of manufacture of the present invention, said porogen is present at between 1 and 40 wt. % in the resin precursor.

Also described herein is a laminate comprising:
(I) at least one layer of a porous polymeric material, said porous polymeric material, as defined herein, comprising at least one polymer comprising a carbon nanoparticle covalently bonded to a polyurethane moiety; and
(II) a layer comprising a resin precursor comprising a porogen, wherein said resin precursor is used for making said porous polymeric material. Thus, said laminate is an intermediate in the production of a layered porous polymeric material or the type described herein.

Said surface used in the aforementioned solvent casting technique may be a planar surface or a rounded surface. The suspension may be coated on said surface by dipping said surface into the aforementioned suspension and/or by pouring, spraying, painting and/or daubing said suspension thereon, and additionally relying on gravity and/or surface tension of said suspension to provide an even layer. In one embodiment, said surface is a planar surface wherein said suspension is coated thereon in a layer, prior to coagulation and curing, as well as leaching of the porogen from said layer. In another embodiment, said surface is a rounded surface comprised on the end of a rod, wherein the end of said rod is dipped in said suspension to form a layer thereon, prior to coagulation and curing, as well as leaching of the porogen from said layer. The solvent casting technique may be repeated n times for each of these embodiments, each time over the previously formed layer of porous polymeric material, results in a porous polymeric material having n layers, wherein n is as defined herein.

Optionally, the resulting porous polymeric material having from one to 1+n layers may be triturated (i.e. washed/extracted) to remove any remaining porogen, solvating agent starting materials and/or any by-products from the formation of said porous polymeric material (e.g. solvated porogen). Preferably, washing is performed in water, ethanol or methanol, or a combination thereof. More preferably, washing is performed in water. Washing is preferably carried out over a period of at least 1 hour, more preferably over a period of 1 day to 1 month, even more preferably over a period of 3 days to 2 weeks, optionally using several cycles of fresh water, ethanol or methanol, or a combination thereof or by Soxhlet extraction.

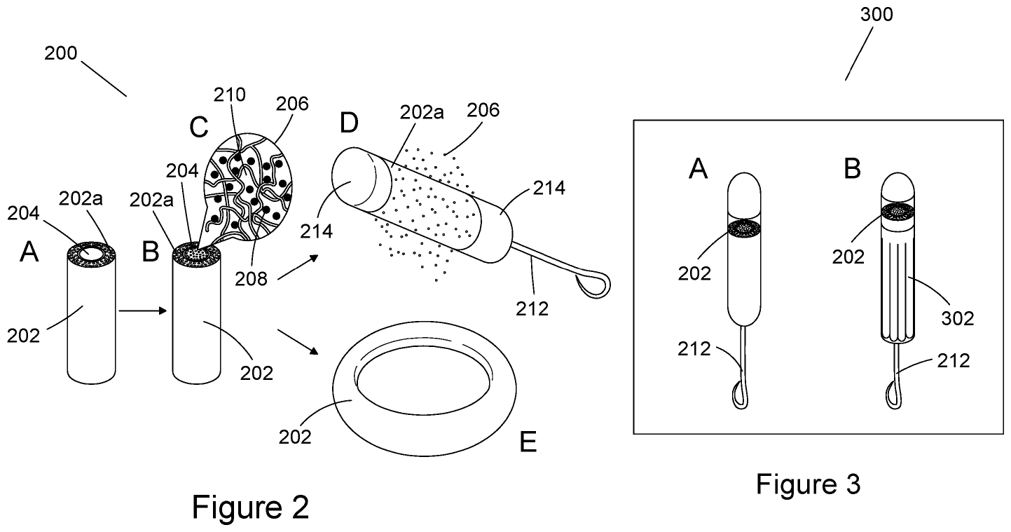

The resulting porous polymeric material having from one to 1+n layers may be directly used in the drug delivery device or a part thereof, or may be cut, bent or folded into a shape suitable for use therein (i.e. a shape which has a space or reservoir in which a drug may be comprised, FIGS. 2 to 4). In a preferred embodiment of the device of the present invention, said device comprises more than one layer of porous polymeric material.

Thus, said porous polymeric material, irrespective of whether formed by solvent casting or not, may be used to form the drug delivery device or a part thereof. In one embodiment of the device of the present invention, said device comprises a wall separating an internal space of said drug delivery device from the outside of said drug delivery device, wherein said internal space is suitable for containing a drug therein and said wall comprises said porous polymeric material. Thus, said porous polymeric material, and hence said wall, is suitable for allowing said drug to be released (i.e. delivered) from said internal space. Preferably, said wall comprises from one to 1+n layers of porous polymeric material, wherein said layers are formed, for example, according to the aforementioned solvent casting technique.

In one embodiment, the porous polymeric material changes the dimensions of the pores therein upon exposure to temperatures above ambient (25° C.) temperature due to thermal conductivity of said material. Thus, after placing the drug delivery device into a body cavity, the size of the pores may change from that which does not allow drug release to that which does allow drug release.

Said device may additionally comprise further materials such as a shape-memory material or superelastic material. A shape-memory material is a material that responds to a change in its environment (e.g. a temperature change) by undergoing a material property change. A superelastic material is a material that exhibits elasticity in response to an applied stress (i.e. it is pseudoelastic). Said shape-memory material may also be a superelastic material and vice versa. Said shape-memory material is preferably a shape-memory polymer or a shape-memory alloy while said superelastic material is preferably a superelastic polymer or a superelastic alloy. A polymer having shape-memory and/or superelastic properties is preferably selected from the group consisting of a polyurethane, a polyurethane comprising ionic or mesogenic components made from a prepolymer, a block copolymer of polyethylene terephthalate (PET) and polyethyleneoxide (PEO), a block copolymer containing polystyrene and poly(1,4-butadiene), and an ABA triblock copolymer made from poly(2-methyl-2-oxazoline) and polytetrahydrofuran. An alloy having shape-memory and/or superelastic properties is preferably selected from the group consisting of a copper-aluminium-nickel and a nickel-titanium (nitinol) alloy. More preferably, said shape-memory material is also a superelastic material which is a nickel-titanium alloy. Said material having shape-memory and/or superelastic properties may be embedded within the porous polymeric material of said device, or encapsulated within an internal space of said device. In the embodiment of the drug delivery device of FIG. 4, the shape-memory material is a shape-memory and superelastic alloy (e.g. nitinol) which is encapsulated within the internal space of a pessary ring (hollow toroid), preferably in the form of a wire. In this way, the pessary ring is folded prior to insertion into the vagina (which may be achieved by e.g. placing the folded ring into a tubular dispenser from which the folded ring is dispensed, once inserted into the vagina), but upon removal of the forces keeping it folded (e.g. by dispensing the folded ring from the tubular dispenser into the vagina) it opens to a toroidal structure (FIG. 7), following which a drug which is also encapsulated within the internal space of said pessary ring may be delivered to the vagina and vaginal walls.

The present invention also relates to a charged drug delivery device (i.e. a drug delivery device, as defined herein, which is charged or loaded with said drug) and to a method of manufacture thereof. Said charged drug delivery device comprises:
(a) a drug delivery device as defined herein; and
(b) a drug, wherein said drug is encapsulated by the porous polymeric material comprised in said drug delivery device.

Said drug is preferably selected from the group consisting of: a cannabis-based product for medicinal use (CBPM), Indometacin, Ketoprofen, Meloxicam, Naproxen, Ibuprofen, Diclofenac (e.g. diclofenac sodium, diclofenac potassium), Mefenamic acid, Aspirin, Codeine phosphate, Tramadol, Dihydrocodeine, Buprenorphine, Co-codamol, Fentanyl, Capsaicin, Gabapentin, Pregabalin, Clonidine, melatonin, Oxytocinon, Estrone, Estriol, Estradiol, Estetrol, Progesterone (including norethisterone, medroxyprogesterone, utrogestan (micronized progesterone), gemstone crinone, dydrogesterone, levnorgestrel, drospirenone, norgestrel), Cycloprogynova (estrodiol with norgestrel), Cyproterone acetate, Tranexamic acid, Letrozole, Tamoxifen, Clomifene citrate, Raloxifene, Misoprostol, Prostaglandin E2, Hydrocortisone, Dexamethasone, Prednisolone, Methylprednisolone, Ondansetron, Clonidine, Gonadorelin, Choriogonadotropin alfa, Chorionic gonadotrophin, Corifollitrophin alfa, Danazol, Triptroelin, Nafarelin, Promethazine, Acyclovir, Famciclovir, Valaciclovir, Clotrimazole, Fluconazole, Isavuconazole, Itraconazole, Ketoconazole, Fenticonazole, Etoconazole, Lactic acid, Miconazole, Dequalinium, Metronidazole, Nitrofurantoin, Co-amoxiclav, Cefalexin, Ceftriaxone, Clindamycin, Ciprofloxacin, Trimethoprim, Gentamicin, Oxybutynin, Darifenacin, Fesoterodine fumarate, Solifenacin, Trospium, Mirabegron, Tolterodine, Propiverine, Lidocaine, Sodium citrate, Cisplatin, Carboplatin, Paclitaxel, Topotecan, Doxorubicin, Gemcitabine, 5-fluorouracil, Azathioprine, Mercaptopurine, 6-thioguanine, Methotrexate, Probiotics, (including e.g. bacterial cultures comprising Lactobacillus acidophilus, Lactobacillus bifidobacterium and/or Lactobacillus rhamnosus), Enfurvitide, Dolutegravir, Elvitegravir, Raltegravir, Etravirine, Nevirapine, Efavirenz, Rilpivirine, Abacavir, Lamivudine, Dolutegravir, Didanosine, Emtricitabine, Mifepristone, Dinoprostone, Carbetocin, Carbeprost, Misoprostol, Gemeprost, Bupivicaine, Levobupivicaine, Mepivacaine, Oxybuprocaine, Prilocaine, Proxymetacaine, and Topivicaine, or a salt of any of said drugs.

More preferably said drug is a CBPM comprising an anti-inflammatory cannabinoid, even more preferably a CBPM comprising a cannabinoid selected from the group consisting of cannabidiol (CBD), cannabigerol (CBG), Δ⁹-tetrahydrocannabinol (THC), 1′,1′-dimethylheptyl-delta-8-tetrahydrocannabinol-11-oic acid (ajulemic acid), 1,1-dimethylheptyl-11-hydroxy-tetrahydrocannabinol (HU-210), [(1S,2S,5S)-2-[2,6-dimethoxy-4-(2-methyloctan-2-yl)phenyl]-7,7-dimethyl-4-bicyclo[3.1.1]hept-3-enyl]methanol (HU-308), (11R)-2-methyl-11-[(morpholin-4-yl)methyl]-3-(naphthalene-1-carbonyl)-9-oxa-1-azatricyclo[6.3.1.04,12]dodeca-2,4(12),5,7-tetraene (WIN55,212-2), dimethylbutyl-deoxy-Δ⁸-THC (JWH-133), and a combination thereof. Yet more preferably, said drug is a CBPM comprising an anti-inflammatory cannabinoid selected from the group consisting of cannabidiol (CBD), cannabigerol (CBG), tetrahydrocannabinol (THC), and a combination thereof.

Alternatively, said drug is more preferably a non-steroidal anti-inflammatory drug (NSAID), even more preferably a NSAID selected from the group consisting of diclofenac, ibuprofen, aspirin, naproxen, flurbiprofen, mefanamic acid, meclofenamic acid, flufenamic acid, and combinations thereof.

Said charged drug delivery device may comprise one or more drugs, wherein each drug is independently selected from the aforementioned list. More preferably, said charged drug delivery device comprises one or more drugs, wherein each drug is independently selected from the group consisting of a CBPM or a NSAID, even more preferably wherein said CBMP is CBD, CBG or THC and said NSAID is diclofenac, ibuprofen, aspirin, naproxen, flurbiprofen, mefanamic acid, meclofenamic acid or flufenamic acid. In the embodiment of Example 7, said drug is diclofenac.

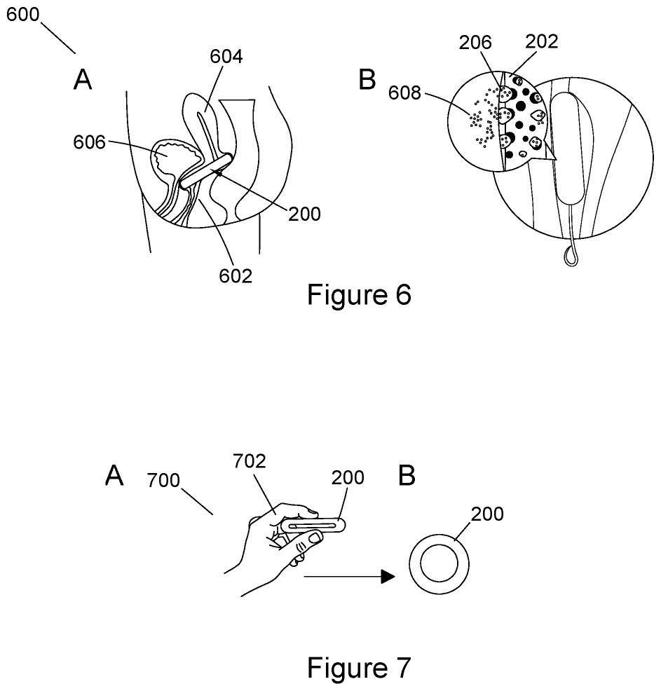

In one embodiment of the charged drug delivery device of the present invention, said drug is dispersed in a hydrogel matrix encapsulated by said drug delivery device. Hydrogels can be selected to respond to stimuli: pH, temperature, ultrasound, electric field, enzymes, and light. Various systems use intelligent hydrogel, supramolecular hydrogel or polymer hydrogel scaffolds and targeted drug delivery. They provide spatial and temporal control over the release of various therapeutic agents, including small molecule drugs, macromolecular drugs and cells, minimising the inconvenience of conventional drug delivery. Compared to nanocapsules, micelles and liposomes, hydrogels exhibit higher drug loading capacity (up to 50% loading efficiency). The hydrogel can exhibit biodegradability, biocompatibility, response to stimuli and high mucoadhesion. The hydrogel may be selected for its specific mucoadhesive properties that allow the absorption and retention time of the drug to be modified according to the type of treatment. In this way, the availability of the drug at the target mucosa can be, for example, increased by selecting a hydrogel having mucoadhesive properties which improve and facilitate the absorption of drugs. For example, when the drug delivery device is a tampon inserted into the vagina, the hydrogel diffuses therethrough and changes the structure of vaginal fluid, such that the viscosity of the vaginal mucus will change, and the hydrogel will gradually adhere to the vaginal mucosa, increasing drug delivery (FIG. 6B).

Said hydrogel is a water-insoluble 3D crosslinked hydrophilic polymer. Said hydrogel can be either hydrophilic (i.e. charged), hydrophobic or a hybrid thereof. The cross-linking density of the network of interpenetrating macromolecular chains of the hydrogel offers adjustable swelling capacity, particle size and porosity, allowing controlled drug loading and diffusion. Preferably said hydrogel is charged and retains the drug by charge-charge interaction, so as to delay release of the drug, and/or swells and generates movement or deformation of its network matrix upon contact with physiological fluids or solvents. More preferably said hydrogel is selected from either the group consisting of natural polymers including: gelatin, chitosan, dextran, hyaluronic acid, alginate, collagen, fibrin, pectin, carrageenan, carboxymethyl chitin, xanthan gum, guar gum and cellulose, or from the group of synthetic polymers including: polyglycolic acid, polylactic acid, hydroxyethyl methacrylate, polyiminocarbonates, polyethylene glycol diacrylate/methacrylate, polyvvinyl alcohol (PVA), polyvinyl pyrrolidone, polyethylene glycol, polyethene imine, polymethacrylate, polyvinyl acetate, polyacrylic acid or polyacrylate, polymethyl methacrylate and carboxymethyl cellulose, or from a charged form of the aforementioned hydrogels.

More preferably, said hydrogel is a cross-linked PVA or a cross-linked polyacrylate such as a Carbopol® (e.g. Carbopol® 674, 676, 690, 691, 940 Aqua 25, Aqua30, Aqua CC, EDT2623, EDT2691, EZ-2, EZ-3, EZ-4, EZ-5 or EC-1 polymer). In the embodiment of Example 7, said hydrogel is a Carbopol® 940.

Preferably, the hydrogel matrix is preferably delivered, together with said drug, from the internal space of said drug delivery device to the outside of said drug delivery device, via the pores in the porous polymeric material comprised in the device of the present invention (FIG. 6B). Thus, said hydrogel is more preferably poroelastic with respect to the pores in the porous polymeric material comprised in the device of the present invention, as defined herein.

The hydrogel can be synthesised chemically, physically, ionically or radically. This is a simple one-pot, sol-gel gelation process but may comprise several reaction steps depending on the chemical composition of the hydrogel. Hydrogels are easy to synthesise and facilitate the incorporation of active substances.

In order for the drug to be dispersed through the hydrogel matrix it may be dissolved in an aqueous or organic solution under controlled physical parameters (e.g. temperature, pH), wherein the physicochemical parameters used depend on the drug and the desired release rate. Once the drug solution is homogeneous, the hydrogel reagents are gradually added and mixed into the solution at a fixed pH and temperature. The hydrogel network will form with the drug dispersed therethrough, by absorption of the drug solution in the matrix thus formed and/or encapsulation of the drug in the matrix as it forms. The final product of drug dispersed in hydrogel can be used with the drug delivery device of the present invention in wet form (i.e. drug encapsulated in wet hydrogel) or in dried form (i.e. drug encapsulated in a dry macromolecular matrix) by encapsulation therein (FIGS. 2 and 4).

It will be appreciated that additives, other than or in addition to said hydrogel, which are suitable for formulating a drug may be included in the charged drug delivery device as defined herein, particularly additives which modulate rate of delivery (i.e. release) of said drug from said drug delivery device. For example, the drug may be comprised in a formulation which is a composition, or said drug may be comprised in nanocapsules, micelles or liposomes, or said drug may be bonded to nanoparticles.

The present invention also relates to a kit-of-parts comprising:
(a) a drug delivery device as defined herein or manufactured according to the method as defined herein; and
(b) a drug for encapsulation in the porous polymeric material comprised in said drug delivery device. Preferably, said drug is as defined herein. Thus, the kit-of-parts provides the necessary parts for the drug delivery device to be charged by the user, prior to administration of the resulting charged drug delivery device to a human or animal subject.

In one embodiment, the drug included in said kit-of-parts may be dispersed in a hydrogel matrix, wherein said hydrogel matrix is as defined herein. Alternatively, the kit-of-parts may comprise:
(a) a drug delivery device as defined herein or manufactured according to the method as defined herein;
(b) a drug for encapsulation in the porous polymeric material comprised in said drug delivery device; and
(c) a hydrogel, as defined herein.

In such an embodiment, the kit-of-parts provides the necessary parts for the drug to be dispersed in the hydrogel by the user, and the delivery device to be charged with said dispersion, prior to administration of the resulting charged drug delivery device to a human or other animal.

The present invention also relates to a charged drug delivery device (i.e. a drug delivery device, as defined herein, which is charged or loaded with said drug) for use in administering a drug, wherein said charged drug delivery device is as defined herein. Administering a drug means administering a drug to a human or other animal, more preferably a human or other mammal, even more preferably a human female of reproductive age. Similarly, the present invention also relates to use of said charged drug delivery device or said kit-of-parts for the manufacture of a medicament for administration to a human or other animal. Analogously, the present invention also relates to a method of administering said charged drug delivery device to a human or other animal, as well as to said charged drug delivery device for the treatment of disease in a human or other animal.

### EXAMPLES

All materials, methods and examples described herein detail specific examples that fall within the scope of the present invention and support the understanding thereof. In addition, this disclosure is illustrative only, so is not limited to any particular methods and experimental conditions described. As such methods and conditions may vary and it is not intended to be limiting in any way. Although alternative methods and materials similar or equivalent to those described herein can be used in the practice or testing of the present disclosure, suitable methods and materials are described below.

### Example 1 Synthesis of Polymeric Material Resin Precursor (Polymer Solution) for Use in the Manufacture of a Drug Delivery Device

A. Synthesis of Graphene Oxide Functionalised with Amine Groups

3 g of graphene oxide (Sigma) (graphene oxide comprises epoxide, hydroxyl and carboxylic acid groups and it was the carboxylic acid groups which are most relevant in this example) was dispersed in toluene using ultrasonication for 30 minutes, cooled to 4° C., and 3 mL concentrated sulfuric acid added drop-wise and the mixture continuously stirred for 40 minutes. Then 3 g of sodium azide was added to the mixture gradually for 30 minutes while continuously stirring. The mixture was then stirred for a period of 12 hours at 60° C., cooled down, diluted with distilled water, filtered and dried under vacuum at 65° C. for 12 hours thereby to produce an intermediate product. 2 g amine of the intermediate product was then dispersed in N,N′-dimethylformamide (DMF) and reduced with 50 mL hydrazine at 70° C. for 12 hours, filtered, washed with deionized water, and dried under vacuum at 65° C. for 10 hours thereby to produce amine functionalised graphene oxide.

### B. Synthesis of a Functionalised Graphene Oxide-Polycarbonate Copolymer (Polymeric Material Resin Precursor)

5 g amine functionalised graphene oxide of Example 1A and 55 g 2000 MW polycarbonate polyol (Sigma) were added to a 500 ml flask containing a mechanical stirrer and nitrogen inlet. The mixture was heated to 80° C. at which point the functionalised graphene oxide dissolved in the polycarbonate polyol. The mixture was then cooled to 40° C., at which point 16 g 4,4′-methylenebisphenyl isocyanate (MDI) was added to the mixture and the temperature maintained at 75 to 85° C. for 90 minutes thereby to form a pre-polymer. 300 g of dimethylacetamide or dimethyl sulfoxide (DMSO) was then added to the pre-polymer over one hour allowing the temperature to cool to 40° C. thereby to form a pre-polymer solution. A mixture of ethylenediamine and diethylamine in the weight ratio of 7.6:1 as a 3.3% w/v dimethylacetamide solution was then added dropwise to the pre-polymer solution at 40° C. over one hour thereby to create a solution of functionalised graphene oxide-polycarbonate copolymer comprising urea and urethane linkages in dimethylacetamide or DMSO. 3 g of 10% w/v butanol in dimethylacetamide was then added to the solution of functionalised graphene oxide-polycarbonate copolymer to neutralise any excess isocyanate thereby to produce a neutralised functionalised graphene oxide-polycarbonate copolymer solution (Hastalex® resin precursor).

The final solids content was 23% w/w. The viscosity of the neutralised functionalised graphene oxide-polycarbonate copolymer solution was 3850+/−96 mPas. The density of the neutralised functionalised graphene oxide-polycarbonate copolymer solution was 1.186+/−0.010 g/mL.

### C. Formation of a Suspension of Functionalised Graphene Oxide-Polycaprolactone Copolymer (Polymeric Material Resin Precursor) Comprising Porogen

A functionalised graphene oxide-polycaprolactone copolymer (BioHastalex® resin precursor was made according to Example 1A and 1B, by replacing 2000 MW polycarbonate polyol (Sigma) with polycaprolactone oxydiethylene ester, average Mn˜2,000; Sigma-Aldrich catalogue number 189421). This functionalised graphene oxide-polycaprolactone copolymer was centrifuged and degassed at 209.4395 rad/s (2000 rpm) for 3 minutes and 146.6077 rad/s (1400 rpm) for 2 minutes respectively, to remove any bubbles and ensure the solution was homogenous in consistency. The resulting polymeric material resin precursor suspension was then used in the following Examples.

### Example 2 Method of Manufacture of a Drug Delivery Device from a Polymeric Material Resin Precursor Suspension

### A. Coating Step

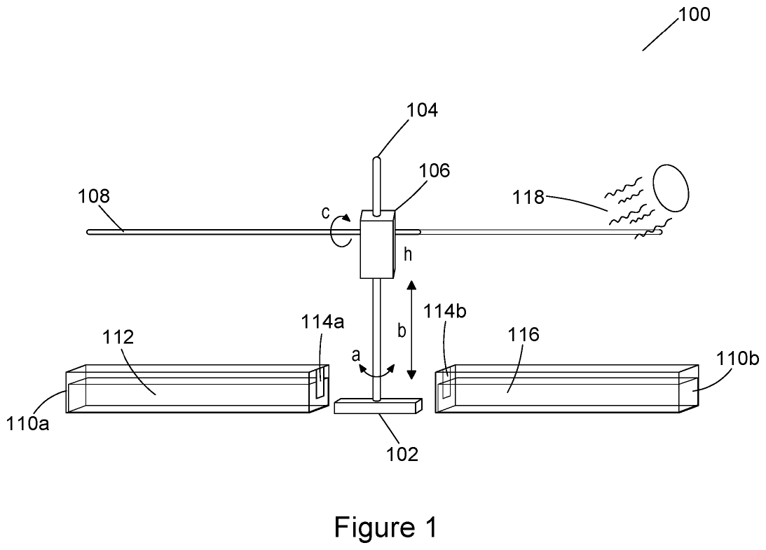

Referring to FIG. 1, an instrument 100 is illustrated, by which the present invention may be made or fabricated. The instrument 100 comprises a stand 102, 104, having a foot 102 for holding a central pillar 104 in a substantially vertical position relative to the foot 102, the central pillar 104 being rotatable about a vertical axis a, e.g. 180°. The central pillar 104 has a template holder 106 for receiving and holding a device template 108 via a template securing means (not shown), the template holder 106 being vertically movable b about the central pillar 104. The device template 108 may also be rotatable about a horizontal axis c. A template holder securing means (not shown) enables the template holder 106 to be secured to the central pillar 104 at a desired height h relative to the foot 102.

The device template 108 is shown in FIG. 1 as a rod, for example a steel rod of which may be about 2 mm diameter, but it will be appreciated that other template designs, for example a ring or plate (e.g. glass plate) may suitably be substituted.

The drug delivery device may be made in several ways, two of which are herein described.

The device template 108 is held in, and lowered by, the template holder 106 towards a first container 110a holding a suspension 112 of Example 1C comprising polymeric material resin precursor (polymer solution) and porogen homogenously suspended therein. This is achieved by releasing the template holder securing means (not shown) and adjusting the height of the template holder 106 by sliding the template holder 106 along the central pillar 104 towards the foot 102, thus moving the template 108 closer to the container 110 of suspension 112. The template 108 is then dipped into the suspension 112 held in the first container 110a. From FIG. 1 it is shown that the first container 110a has a notch 114a in the side wall nearest to the stand 102, 104 enabling the template 108 to be lowered further into the suspension 112 of the first container 110a to fully submerge the template 108. Once in the desired position the template holder 106 may be held in place via the template holder securing means (not shown). Once fully coated in the suspension 112, the template 108 is then removed from the suspension 112 held in the first container 110a by reversing the step described above.

### B. Coagulation and Curing Step

The template 108 now coated in suspension 112 is moved towards the second container 110b by rotating the central pillar 104 about the vertical axis a until the template 108 coated in suspension 112 is positioned above the second container 110b (e.g. this may be about 180° when the containers 110a, 110b are positioned substantially opposite one another either side of the stand 102,104. The template 108 is then lowered by the template holder 106 in a similar way to that described in the coating step described herein, but this time towards the second container 110b holding a curing solvent 116 (e.g. water at 60° C.). The template 108 coated in suspension 112 is then dipped into said solvent for 1 minute (±15 seconds) to coagulate and cure at least the surface of the polymer solution (or polymeric material resin precursor). From FIG. 1 it is shown that the second container 110b has a notch 114b in the side wall nearest to the stand 102, 104 enabling the template 108 to be lowered further into the curing solution 116 of the second container 110b to fully submerge the template 108. Once in the desired position the template holder 106 may be held in place via the template holder securing means (not shown). Once cured, any excess moisture (from the curing solution) is removed from the now coagulated and cured resin (or polymeric material) still coating the template 108.

The template 108 may be a glass sheet, in which case the suspension is coated on one side of the glass plate to form a uniform layer and cured as described herein.

To add a further layer the procedure is repeated by coating said suspension on the coagulated and cured porous polymer previously formed, and subsequently coagulating and curing as per the step described above. The number of additional layers, and thus the number of repetitions of coating and coagulating and curing depends upon the desired rate of release of a drug (once loaded into the fully formed device). Preferably this coating and coagulating and curing process may be repeated 8-15 times, respectively resulting in 8-15 layers of porous polymeric material.

Alternatively, the template 108 at least partially coated in suspension 112 may be cured by drying the polymeric material resin precursor). Here the template 108 at least partially coated in suspension 112 is raised out of the first and/or second containers 110a, 110b and held by the template holder 106 fixed in position by template holder securing means (not shown) at a desired height h on the central pillar 104 adjacent a heat curing source 118. Preferably, the template 108 at least partially coated in suspension 112 is heat cured (dried) by heating at about 60° C. for 3-6 hours.

FIG. 5 shows two scanning electron microscopy (SEM) images of a porous polymeric material 202a according to certain aspects of the invention, for use in a drug delivery device.

Referring now to FIG. 5A, the SEM image shows a 3D porous scaffold of the polymeric material, with a given porosity fabricated using porogens having a size of about 60 μm.

Referring now to FIG. 5B, the SEM image shows scaffold a 3D porous scaffold of the polymeric material with a given porosity fabricated using porogens having a size of about 100 μm.

As can be seen from each of the SEM images of FIGS. 5A and 5B, the pore size and distribution may be irregular.

### C. Shaping or Moulding the Device

The resulting cured resin product may be subsequently shaped or moulded into the desired shape and size of the final drug delivery device. How this is achieved somewhat depends on which of the methods, as described herein, has been used to prepare the resulting cured resin product.

### Rod Template

Where a rod template/mould is used, once the polymeric material has been cured and coagulated thereon, the template is removed from inside the cured resin product (e.g. cured polymeric material) leaving a hollow tubular shell.

The ends of the tube may then be sealed or otherwise capped using resin material, which may be the same or different to the polymeric material used for the body of the device. Either the ends of the tube or the caps are secured in place by means of further polymer solution (e.g. BioHastelex® resin precursor) which is subsequently cured.

Alternatively, the tube may be curled upon itself so that each of the open ends of the tube meet (i.e. to form a ring shape). Each of these open ends are sealed to one another by means of further polymer solution (e.g. BioHastelex® resin precursor) which is subsequently cured.

### Plate Template

Where a plate template/mould is used, once the polymeric material has been cured and coagulated thereon, the resulting sheet is lifted off the plate, rolled into a tube and the sides secured with further polymer solution (e.g. BioHastelex® resin precursor) which is subsequently cured to form a drug delivery device for use as a pessary.

### Ring Template

Where a ring template/mould is used, once the polymeric material has been cured and coagulated thereon, the cured product (i.e. polymeric material) is removed from the template leaving a half-toroidal shell. Two of these half-toroidal shells make up a completed toroidal (ring-shaped) drug delivery device. One of the half-toroidal shells is superimposed on the other and the two halves are secured together with further resin (e.g. BioHastelex® resin precursor) which is subsequently cured to form said ring-shaped drug delivery device for use as a toroidal pessary

It will be appreciated that at least one part of the shell (tube or half-toroidal shell) may remain open until a drug and/or additional additives and/or shape memory material has been inserted into the internal space of the tube.

After fabrication, the tube is washed in water until organic solvents and by-products are no longer detectable and dried at room temperature (25° C.).

### Example 3 Control of Mechanical Properties by Varying the Coagulation and Curing Step

The mechanical properties of materials prepared by various methodologies were analysed by measuring the tensile strength. To do so, a polymer film was fabricated with 1 to 3 layers of BioHastalex® resin precursor (polymer solution) (5 mL of resin precursor in each layer) and the stress at break was compared between formulations. A summary of conditions used and tensile stress at break values is presented in Table 1.

TABLE 1
A summary of the conditions used for the preparation of
polymer film and tensile stress at break values, measured
with a tensile strength machine (AML Instruments) according
to ISO 527-1: 2019(en); n = sample size.

| | Number of | | Tensile stress | |
|---|---|---|---|---|
| Polymer solution | layers | Cured in | at break, N/mm² | n |
| BioHastalex | 1 | 60° C. oven | 60.8 ± 4.8 | 3 |
| | 1 | approximately | 3.8 ± 1.0 | 3 |
| | | 25° C. water | | |
| | 1 | approximately | 6.6 ± 0.2 | 4 |
| | 2 | 90° C. water | 19.5 ± 1.5 | 3 |
| | 3 | | 21.0 ± .5 | 4 |
| BioHastalex + | 1 | | 4.2 ± 0.4 | 4 |
| 5 w/w % NaHCO₃ | | | | |
| porogen | | | | |

As the data show, the variations in the method of fabrication of the result in differences in mechanical properties. Curing the BioHastalex® polymer solution at 60° C. in an oven resulted in the strongest material made of 1 layer of polymer resin. Curing in 90° C. water led to the formation of a greater amount of crosslinked polymer network in comparison to materials prepared in room temperature (25° C.) water. The addition of porogen NaHCO₃ increases the porosity but reduces the mechanical strength. However, the use of several layers of resin precursor can significantly improve the characteristics of the final polymeric material (and hence the porous polymeric material), resulting in increased tensile stress at break. Therefore, the tensile strength data shows how conditions for material fabrication can be tailored to create a product with mechanical properties suitable for the use as a suppository, e.g. a vaginal pessary, while changing the rate of drug release with porosity.

### Example 4 Fabrication of Drug Delivery Devices and Charged Drug Delivery Devices

FIG. 2 illustrates how a drug delivery device 200 is fabricated from a polymeric shell and actives and/or drug delivery additives into its final form. Parts similar to features described herein are accorded the same reference number.

Referring now to FIG. 2A, a cross-sectional perspective diagrammatic view of a portion of the drug delivery device 200 prior to the device being charged with drug is shown. The drug delivery device 200 comprises an external wall (or outer shell) 202 formed from polymeric material 202a, the external wall 202 defines an internal space 204. Here the internal space 204 is shown as empty. The porous nature of the polymeric material 202a is shown by the cross-section.

Referring now to FIG. 2B, a cross-sectional perspective diagrammatic view of a portion of the drug delivery device 200 once it has been charged with drug is shown. The drug delivery device 200 comprises an external wall (or outer shell) 202 formed from polymeric material 202a, the external wall 202 defines an internal space 204. Here the internal space 204 is shown as containing a drug and, optionally, an additive. The porous nature of the polymeric material 202a is shown by the cross-section.

Referring now to FIG. 2C, a close-up diagrammatic view of the active for delivery 206, i.e. drug loaded in to the internal space 204 is also shown. Here the active for delivery 206 comprises a drug 208 and an additive 210. Here the additive 210 is shown as a matrix by virtue of the lines. The additive/matrix 210 may include a hydrogel for encapsulating the drug 208.

Referring now to FIG. 2D, a perspective diagrammatic view of the drug delivery device 200 according to certain aspects of the invention, namely a tampon-like device (TLD) is shown. The drug delivery device 200 comprises an external wall (or outer shell) 202 forming the body of the device and a tail portion (e.g. a length of string) 212, suitable for assisting the insertion and retrieval of the device. Here at least a portion of the body of the device 202 is formed from a porous polymeric material 202a, shown as a darker central band. The active for delivery 206 (e.g. a drug 208 and, optionally, an additive 210) is shown as released from this central portion of the external wall 202 formed from porous polymeric material 202a. The ends 214 of the device 200 are shown as enclosed such that the active for delivery 206 is not released through this part of the device 200 (i.e. is not made from a porous polymeric material.

Referring now to FIG. 2E, a perspective diagrammatic view of the drug delivery device 200 according to certain aspects of the invention, namely a ring pessary 216 is shown. The drug delivery device 200 comprises an external wall (or outer shell) 202. In contrast with FIG. 2D, the mode of drug release is not shown, but the ring pessary 216 comprises, at least in part, the portion shown in FIG. 2A and, when charged with drug and, optionally an additive, FIG. 2B and so is, at least in part, formed from polymeric material 202a that would enable the release of the active for delivery 206.

FIG. 3 illustrates a drug delivery device 200 according to certain embodiments of the invention.

Referring now to FIG. 3A, there is shown a diagrammatic exploded view of the drug delivery device as a pessary or tampon-like device (TLD). The drug delivery device comprises an external wall (or outer shell) 202 forming the body of the device and a tail portion (e.g. a length of string) 212, suitable for assisting the insertion and retrieval of the device. Here, while mode of drug release is not shown, the whole device is made from the porous polymeric material 202 as indicated by the whole device being a darker grey colour. The cross-section of the device shows the internal space containing the active for delivery surrounded by the external wall 202.

Referring now to FIG. 3B, there is shown a tampon or tampon-like device 302 (e.g. comprising cotton or similarly suitable absorbent material) comprising the drug-delivery device and a tail portion (e.g. a length of string) 212, suitable for assisting the insertion and retrieval of the tampon comprising the device. The drug delivery device also comprises an external wall (or outer shell) 202 forming the portion of the device configured for drug delivery. The cross-section of the device shows the internal space containing the active for delivery surrounded by the external wall 202. Here the device is shown positioned at the tapered end of the tampon 302. It will be appreciated that the device may be positioned at any point of the tampon 302 without departing from the invention.

FIG. 4 illustrates a drug delivery device 200 according to certain aspects of the invention.

Referring now to FIG. 4A, shows a diagrammatic perspective view of a ring-shaped (toroidal) pessary, wherein a portion of the device 200 is depicted as transparent to show a cross-section thereof.

Referring now to FIG. 4B, a close-up view of the cross-section of the device 200 is shown. The device 200 comprising the internal space 204 containing the active for delivery 206 and surrounded by the external wall 202. The active for delivery 206 is shown as released through the external wall 202 formed from porous polymeric material 202a. Also shown is a shape-memory material 402 positioned in the internal space 204 of the device 200. While here the shape-memory material 402 is shown to be centrally positioned and surrounded by the active for delivery 206, other arrangements of the shape-memory material within the device 200, preferably within the internal space 204 of the drug delivery device 200, may suitably be employed without departing from the invention.

Nano-fabrication and micro-fabrication of porous scaffolds made from polymeric material, as herein described, may be performed. Advantageously, the porous 3D scaffold enables controlled release of an active for delivery, optionally the controlled release of a drug from the drug delivery device. Release of the drug or active through the 3D scaffold may be further controlled by forming the 3D scaffold into a tampon-like shape or pessary ring-like shape, as per the device described herein.

### Example 5 Drug Delivery Device in Use

Referring generally to FIG. 6 there is shown a drug delivery device 200 according to certain aspects of the invention, when in use.

Referring now to FIG. 6A, placement of the drug delivery device 200, here a pessary, in situ is shown. The drug delivery device 200 is inserted into the vagina 602 and positioned such that is pressed against the uterus 604 and positioned substantially opposite the bladder 606. It is to be appreciated that the device may also or alternatively press against the bladder 606 and/or other internal organs (not shown) so as to reposition said organs and stop them from pressing on the vagina 602. Advantageously, the device 200, by pressing against the uterus 604 and/or bladder 606 and/or other internal organs (not shown), provides support to the internal muscles that would otherwise support and hold said organs in position, but which may be weakened for example through age or post-partum, besides delivering a drug when charged. Provision of internal muscle support by the device 200 thus prevents and/or reverses pelvic floor prolapse or pelvic organ prolapse (POP). Here, drug delivery is not shown and not required for support purposes.

Referring now to FIG. 6B, the delivery of the active 206 (e.g. drug 208 and, optionally, an additive 210) to the site of action from the drug delivery device 200, here a pessary placed in situ is shown. A close-up of the drug delivery device 200 in situ shows that delivery of the active 206 (e.g. drug and, optionally, an additive) is achieved by said active 206 moving from the internal space through the external wall 202 of the device 200, by virtue to the porous polymeric material from which the external wall 202 may be formed. Once the active has exited the device 200 it migrates towards the site of action 608, here shown as the wall of the vagina (and/or the surrounding muscle/tissue) 608 and penetrates the vaginal wall. Once the active has arrived at the site of action 608 the drug initiates a cascade of events via the mechanism and/or mode of action associated therewith.

FIG. 7 shows a drug delivery device 200 according to certain aspects of the invention, in which the device 200 comprises a shape-memory material 402.

Referring now to FIG. 7A, the device 200 is diagrammatically shown in a compressed state. Pressure 702, here shown as a user squeezing the device between their finger and thumb, applied to the device 200 causes the device 200 to deform and take on a new shape as a result of the pressure 702 applied.

Referring now to FIG. 7B, the device 200 is diagrammatically shown in its free or uncompressed state. Once pressure 702 is removed from the device 200, the device may return to its original shape upon exposure to heat, here shown as the ring-shape of the pessary according to certain aspects of the invention.

It will be appreciated that while a ring-shaped pessary 200 is shown, drug delivery devices having alternative shapes may be made.

Advantageously, a drug delivery device comprising shape-memory material may, in addition to delivering a drug, also improve ease of use and insertion (e.g. accuracy and avoiding pain from improper insertion), particularly where the device 200 is self-administered by the user (rather than inserted by e.g. a medical professional into the patient).

### Example 6 Formulation of Drug Composition

Diclofenac (10 mg) was dissolved in 200 μL of a 1 wt. % aqueous solution of carbopol at approximately 25° C. with stirring. A neutralizing agent was added to activate hydrogel formation and the resulting mixture was stirred gently to form a carbopol hydrogel matrix comprising diclofenac dispersed therein.

### Example 7 In Vitro Drug Release Profile of the Drug Delivery Device According to an Embodiment of the Present Invention

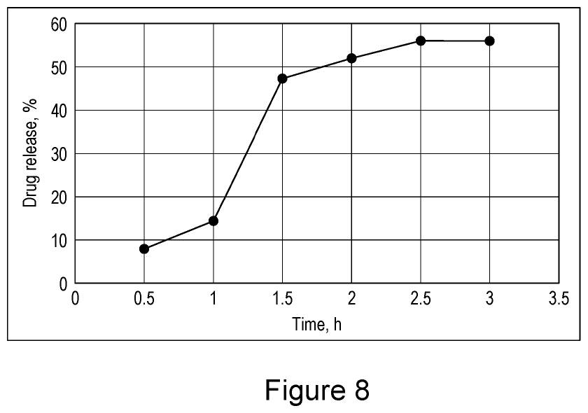

The rate of drug release was assessed by using a vaginal suppository fabricated according to the methods as described herein. The hollow tube was prepared by curing BioHastalex® resin precursor (polymer solution, refer to Example 1C) for 1 min in 70 to 100° C. water to a total of 10 resin layers. Dimensions of the tube used: height 60 mm, 12 mm diameter. After purification of the hollow tube by thorough washing with water for 7 days, it was filled with 10 mg of diclofenac potassium dispersed in 200 μL of 1 wt % of Carbopol® 940 gel, as described in Example 6. The edges of the tube were sealed with an impermeable material to ensure the drug release through the pores of the porous BioHastalex® copolymeric material comprised in the resulting charged drug delivery device. A preliminary experiment on the drug release was carried out in 200 ml of distilled water at room temperature with intensive stirring. 5 mL aliquots of the releasing media were taken every 30 minutes during the first 3 hours of the experiment and replenished with distilled water. Aliquots were analysed by UV-vis spectroscopy at the wavelength of 282 nm and the concentration of diclofenac was measured according to the calibration curve build at the concentration range between 0.001 and 0.01 mg/ml (R²=0.9981). The rate of diclofenac release measured under conditions described is presented in FIG. 8.

The presented data prove that BioHastalex® can be successfully applied as a material for drug release through porous structure. About 55% of the loaded diclofenac was released in the first 3 hours of the experiment, while the complete release was recorded in 2 days. The rate of drug release can be modified by varying the porosity of the BioHastalex® tube as well by using a different material as a filling.

### Example 8 In Vivo Tolerability and Toxicokinetics of TLDED in Sprague Dawley Female Rats-Repeat Application

The aim of this preclinical experimental study was to test the newly developed tampon-like drug delivery/eluting device (TLDDD) using a rat model.

### A. MATERIALS AND METHODS

### Formation of Vaginal Suppositories

Each vaginal suppository (TLDDD) was fabricated according to the methods as described herein. Each vaginal suppository was prepared by curing BioHastalex® resin precursor (polymer solution, refer to Example 1C) for 1 min in 70 to 100° C. water to a total of 10 resin layers, resulting in a hollow tube. Each hollow tube was dimensioned to fit the vagina of nulliparous and non-pregnant adult female rats. After purification of each hollow tube by thorough washing with water for 7 days, a first set of the hollow tubes were left unfilled, a second set of the hollow tubes were each filled with 70 mg of diclofenac potassium, and a third set of the hollow tubes were each filled with 70 mg of cannabidiol (CBD) and the edges/ends of each tube were sealed with an impermeable material to form first TLDDDs, second TLDDDs and third TLDDDs, respectively.

Animals were sourced from a UK-approved designated animal breeding facility, Charles River (UK) Limited. The animals were clinically healthy, laboratory bred and experimentally naive, females were nulliparous and nonpregnant.

Animals were acclimatised for 6 days prior to surgical procedure and 7 days prior to the test item administration from the day of arrival. Upon arrival at the animal facility, animals were examined by the husbandry staff and housed for acclimatisation. During the acclimatisation period all animals were observed for mortality and morbidity at least once a day. The health status of the animals was evaluated in accordance with accepted veterinary practice and animals were confirmed to be healthy and suitable for a study by the Study Director.

### Ethical Statement/Humane Care of Animals

The in-life (in vivo) experimental procedures undertaken during the course of this study complied with all applicable sections of the United Kingdom Animals (Scientific Procedures) Act 1986 (as amended) and the associated Codes of Practice for the Housing and Care of Animals used in Scientific Procedures and the Humane Killing of Animals under Schedule 1 to the Act, issued under section 21 of the Act.

### Experimental Procedure

Each rat was lightly anaesthetised (isoflurane, 2-5%) and a first TLDDD, second or third TLDDD was administered intravaginally. Rats administered with a first TLDDD were assigned to Group 1, rats administered with a second TLDDD were assigned to Group 2, and rats administered with a third TLDDD were assigned to Group 3. Devices were replaced every 24 hours for three days. Animals were sacrificed at around 72 hours.

### Blood Sampling

Animals were anaesthetised (isoflurane, 5%) and blood was collected at termination via cardiac puncture and processed as below:

For clinical chemistry: >1.5 mL were collected into lithium heparin tubes (blood micro tube 1.3 mL, LH 25 I.U./mL) gently inverted few times and mixed using tube rotator for no less than 15 minutes, placed on wet ice until processed for plasma. The blood was centrifuged (18620 g for 5 minutes at around 4° C.) and all the resulting plasma transferred to corresponding labelled tubes prior to being frozen and stored at −29.62° C. to −23.23° C. When sampling for the study was completed, the samples were transferred to the freezer and stored at −78° C. to −79° C. until shipped for analysis. Animals were not fasted prior to sampling.

### Necropsy and Macroscopic Examination

Animals were examined externally and internally. The gross necropsy included examination of:
external: ID verification, general body condition, fur condition, skin condition, eyes, mouth, teeth, nose, anus, external genitalia, external lesions, masses, fluid.
internal: cranial, thoracic, abdominal, and pelvic cavities with their associated organs and tissues.

All abnormalities, if present, were described, recorded, and retained.

### Organ Weights

The following tissues were weighed: vagina, liver, spleen, kidney, brain, lung, heart.

### Tissue Retention

Vagina, liver, spleen, kidney, brain, lung, heart was trimmed of fat and connective tissues. Post-weighing tissues were immersed in 10% neutral buffered formalin (NBF) to a ratio >1:20 tissue: NBF in labelled pots. Tissues were stored at room temperature until processed for histology.

### B. TOXICOKINETIC BIOANALYSIS

Sample analysis method: UHPLC—tandem mass spectrometry using electrospray ionisation.

Sample preparation: protein precipitation with acetonitrile.

Clinical pathology: plasma samples were shipped to an approved clinical pathology subcontractor on solid carbon dioxide (dry ice) for clinical chemistry analyses using a Beckman-Coulter DxC 700 AU analyser. Temperature was not monitored in transit.

Clinical chemistry: amounts of the following were assessed: albumin, alanine transaminase (ALT), aspartate transaminase (AST), alkaline phosphatase (ALP), creatinine (CRE), bilirubin, low density lipoprotein (LDL), high-density lipoprotein (LDH), urea, uric acid, cholesterol.

### C. RESULTS

### Mortality Results:

No mortality or moribundity were observed in test item treated animals throughout the experimental period.

### Body Weight and Organ Weight Results:

For rats of Group 1, animal body weights and weight dynamics are presented in Table 2. During the study there was an unremarkable body weight dynamics observed, and there were no notable differences in the mean group body weight changes seen on days 1-4. Table 3 shows the organ weights.

TABLE 2
Group 1 animal body weights: individual and mean values

| | Group 1 | | | | |
|---|---|---|---|---|---|
| | (TLDDD non-loaded, Q24 h × 3 | | | | |
| | intravaginal applications) | | | | |
| Animal ID: | 1 | 2 | 3 | 4 | 5 |
| Day 1 weight (g) | 282 | 291 | 290 | 284 | 280 |
| Day 2 weight (g) | 276 | 293 | 293 | 281 | 278 |
| Day 3 weight (g) | 274 | 292 | 295 | 285 | 281 |
| Termination weight, day 4 (g) | 277 | 290 | 289 | 285 | 275 |
| Weight gain/loss (g) | −5 | −1 | −1 | 1 | −5 |
| Weight gain/loss (%) | −1.8 | −0.3 | −0.3 | 0.4 | −1.8 |
| Group weight gain/loss, | −0.8 (1.0) | | | | |
| [%, mean (SD)] | | | | | |

TABLE 3
Group 1 (TLDDD non-loaded, Q24 h × 3 intravaginal applications) mean organ weights

| | Organ | | | | | | | |
|---|---|---|---|---|---|---|---|---|
| | | Kidney | Kidney | | | | | |
| | Spleen | (left) | (right) | Liver | Vagina | Heart | Lung | Brain |
| Weight | 0.5836 | 1.0640 | 1.0589 | 11.4749 | 2.1647 | 1.1094 | 2.3291 | 2.0057 |
| [g, mean (SD)] | (0.0741) | (0.1086) | (0.0956) | (0.3840) | (0.5918) | (0.0749) | (0.3595) | (0.1001) |

Similar results to those shown in Tables 2 and 3 were also obtained in female Sprague Dawley rats of Group 1 which were each subcutaneously inserted with a first TLDDD (made according to the method for making vaginal suppositories described in Example 8A using BioHastalex® resin precursor or using Hastalex® resin precursor instead of BioHastalex® resin precursor) and monitored for 14 days. Each result obtained using BioHastalex® resin precursor fell within two standard deviations of that obtained using Hastalex® resin precursor, and vice versa.

### Toxicokinetic Study Results:

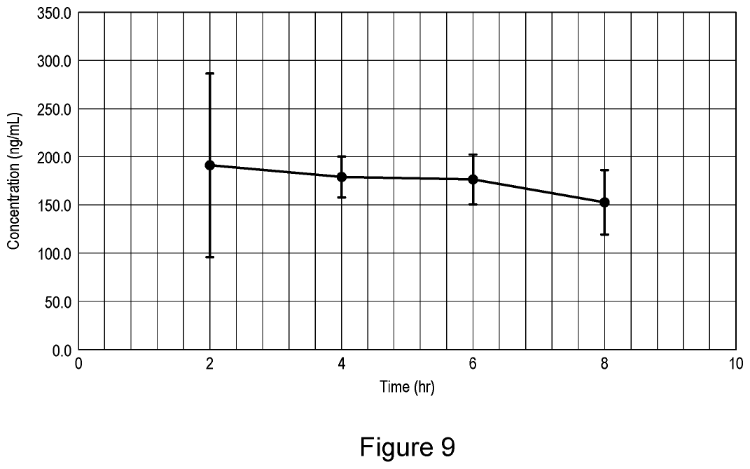

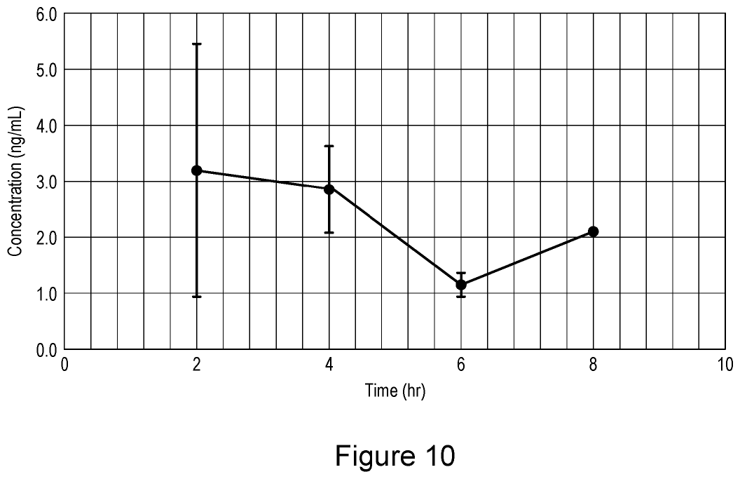

Table 4 and FIGS. 9 and 10 show the toxicokinetic study carried out with TLDDDs charged both with diclofenac and CBD (i.e. from Groups 2 and 3, respectively). These results show that drugs are released successfully in the vagina. Table 5 shows the biochemistry data.

TABLE 4
Toxicokinetic test results showing mean plasma concentrations
of drug in Group 2 and 3 rats over time

| Group 2 (TLDDD charged | | Group 3 (TLDDD charged | |
|---|---|---|---|
| with 70 mg diclofenac) | | with 70 mg CBD) | |
| | Concentration drug | | Concentration drug |
| | in plasma [ng/mL, | | in plasma [ng/mL, |
| Time point | mean (SD)] | Time point | mean (SD)] |
| Pre-dose | <LLOQ | Pre-dose | <LLOQ |
| 2 hr | 191.3 (95.4) | 2 hr | 3.2 (2.3) |
| 4 hr | 178.7 (20.8) | 4 hr | 2.9 (0.8) |
| 6 hr | 176.4 (25.9) | 6 hr | 1.2 (0.2) |
| 8 hr | 152.8 (33.4) | 8 hr | 2.1 (0.0) |

LLOQ = lower limit of quantitation

TABLE 5
Clinical pathology: mean values of amounts of
biochemical markers in Group 1 (TLDDD non-loaded,
Q24 h × 3 intravaginal applications) rats

| Biochemical marker | Mean amount (SD) | | Normal values¹ | |
|---|---|---|---|---|
| Urea | 5.0 (1.3) | mmol/L | — | |
| Total bilirubin | 2.3 (0.4) | μmol/L | 5.3-70.7 | μmol/L |
| ALT | 39 (6) | U/L | 6-114 | U/L |
| ALP | 179 (30) | U/L | 21-367 | U/L |
| Albumin | 38.0 (1.7) | g/L | 29.0-48.0 | g/L |
| AST | 96 (13) | U/L | 37-205 | U/L |
| Total cholesterol | 2.47 (0.30) | mmol/L | 2.22-15.61 | mmol/L |
| Uric acid | 45* (0) | μmol/L | — | |
| LDH | 367 (90) | U/L | — | |
| LDL | 0.90 (0.11) | mmol/L | — | |
| Creatinine | 27.9 (4.6) | μmol/L | 35.4-132.6 | μmol/L |

¹Modified from Loeb, WF and Quimby, FW. 1999. The Clinical Chemistry of Laboratory Animals, 2nd ed. Philadelphia: Taylor & Francis USA.
*The individual values were below the analytical measuring range, these were substituted with the minimal detectable level of 45 μmol/L for calculation purposes.

Similar results to those shown in Table 5 were also obtained in female Sprague Dawley rats which were each subcutaneously inserted with a first TLDDD (made according to the method for making vaginal suppositories described in Example 8A using BioHastalex® resin precursor or using Hastalex® resin precursor instead of BioHastalex® resin precursor) and monitored for 14 days. Each result obtained using BioHastalex® resin precursor fell within three standard deviations of that obtained using Hastalex® resin precursor, and vice versa.

### D. CONCLUSION

The tampon-like drug delivery device (TLDDD) proved to be capable of delivering diclofenac and CBD drugs systemically over 8 hours following intravaginal application, however plasma concentrations of the drugs were generally low and there was no common pattern in drug absorption between individual animals.

There were no clinical signs of local or systemic toxicity observed in any TLDDD-treated animal.

To conclude, in view of the results discussed above, and based on unremarkable clinical observations, lack of morbidity, unremarkable changes in body and organ weights, as well as external and internal macroscopic examination, levels of blood clinical chemistry parameters, thrice repeated intravaginal insertions of a non-loaded TLDDD over a period of 24 hours in Sprague Dawley female rats showed no observed adverse effects of local or systemic acute toxicity related to the TLDDD, which was tolerated well throughout the experimental period of 3 days under the conditions of the study.

1. A drug delivery device comprising a porous polymeric material for modulating the delivery of the drug, the porous polymeric material comprising at least one polymer comprising a carbon nanoparticle covalently bonded to a polyurethane moiety.

2. The drug delivery device according to claim 1, wherein the porogen used for making the pores of the porous polymeric material is a solid material having a particle size of between 65 nm and 1000 μm.

3. The drug delivery device according to claim 1, wherein the porous polymeric material is coagulated and cured in air at between 5° and 90° C. or in water at between 2 and 90° C.

4. The drug delivery device according to claim 1, wherein the porogen used for making the pores of the porous polymeric material is present at between 1 and 40 wt. % in the resin precursor used for making it.

5. The drug delivery device according to claim 1 comprising more than one layer of polymeric material.

6. The drug delivery device according to claim 1 comprising a wall separating an internal space of the drug delivery device from the outside of the drug delivery device, wherein the internal space is suitable for containing a drug therein and the wall comprises the porous polymeric material.

7. The drug delivery device according to claim 1, wherein the drug delivery device is a suppository.

8. The drug delivery device according to claim 1, wherein the drug delivery device is selected from the group consisting of a pessary, tampon, menstrual cup and any other gynaecological device.

9. A method of manufacture of a drug delivery device comprising a porous polymeric material for modulating the delivery of the drug, the porous polymeric material comprising at least one polymer comprising a carbon nanoparticle covalently bonded to a polyurethane moiety, wherein the method comprises the following steps:
(i) mixing the porous polymeric material resin precursor with a porogen to form a suspension;
(ii) forming a layer of the suspension; and
(iii) coagulating and curing the layer formed in step (ii).

10. The method of manufacture according to claim 9, wherein the method comprises additional steps of:
(iv) forming a layer of the suspension on the porous polymeric material;
(v) coagulating and curing the layer formed in step (iv); and
(vi) optionally repeating steps (vi) and (v).

11. The method of manufacture according to claim 9, wherein the porogen has a particle size of between 65 nm and 1000 μm.

12. The method of manufacture according to claim 9, wherein the porogen is present at between 1 and 40 wt. % in the resin precursor.

13. The method of manufacture according to claim 9, wherein coagulating and curing is achieved by exposing the layer formed in step (ii) and/or the laminate formed in step (iv) to air at between 5° and 90° C. or to water at between 2 and 90° C.

14. A charged drug delivery device comprising:
(a) a drug delivery device according to claim 1; and
(b) a drug, wherein the drug is encapsulated by the porous polymeric material comprised in the drug delivery device.

15. The charged drug delivery device according to claim 14, wherein the drug is dispersed in a hydrogel matrix encapsulated by the drug delivery device.

16. A method of manufacture of a charged drug delivery device, wherein the method comprises encapsulating a drug inside the porous polymeric material comprised in the drug delivery device according to claim 1.

17. The method of manufacture according to claim 16, wherein the drug is dispersed in a hydrogel matrix.

18. A kit-of-parts comprising:
(a) a drug delivery device comprising a porous polymeric material for modulating the delivery of the drug, the porous polymeric material comprising at least one polymer comprising a carbon nanoparticle covalently bonded to a polyurethane moiety or manufactured according to the method of claim 9; and
(b) a drug for encapsulation in the porous polymeric material comprised in the drug delivery device.

19. A charged drug delivery device for use in administering a drug, wherein the charged drug delivery device is the charged drug delivery device according to claim 14.
